(19) United States
(12) Patent Application Publication
(10) Pub. No.: US 2022/0330594 A1
(43) Pub. Date: Oct. 20, 2022

(54) METHYLTHIONINIUM AS ENHANCERS OF THE COGNITIVE FUNCTION

(71) Applicant: WisTa Laboratories Ltd., Singapore (SG)

(72) Inventors: Gernot Riedel, Aberdeen, Aberdeenshire (GB); Charles Robert Harrington, Aberdeen, Aberdeenshire (GB); Jochen Klein, Frankfurt (DE); Karima Schwab, Berlin (DE); Claude Michel Wischik, Aberdeen, Aberdeenshire (GB)

(73) Assignee: WisTa Laboratories Ltd., Singapore (SG)

(21) Appl. No.: 17/620,956

(22) PCT Filed: Jun. 29, 2020
(86) PCT No.: PCT/EP2020/068229

§ 371 Date: Dec. 20, 2021

(30) Foreign Application Priority Data
Jul. 1, 2019 (GB) 1909454.9

(51) Int. Cl. A23L 33/10; B65D 25/34; B65D 75/36

(52) U.S. Cl. CPC A23L 33/10; B65D 25/34; B65D 75/36; A61J 1/035

(57) ABSTRACT

The present invention relates generally to nootropic compositions comprising Leuco-methylthioninium acid salts and their uses for cognitive enhancement in normal (non-demented) individuals.

### TECHNICAL FIELD

The present invention relates generally to nootropic compositions and the uses thereof for cognitive enhancement in normal individuals.

### BACKGROUND ART

The use of psychoactive substances to increase the performance of normal, healthy (non-impaired) individuals at work or while studying has been of increasing interest in recent years (Maier et al. 2018). Such substances are sometimes referred to as pharmacological cognitive enhancers (PCEs), nootropics, or smart drugs.

Such substances may be desired for use by students in pursuit of better grades, military personnel who need to remain alert for long missions, and individuals generally keen to better their cognitive performance.

Examples of drugs used for this purpose include methylphenidate and modafinil. Other drugs used generally for the purpose of cognitive enhancement in healthy individuals include caffeine, nicotine, amphetamines, and agents modulating acetylcholine breakdown, or NMDA receptor activities. A variety of traditional herbs, vitamins and supplements have also been suggested. These agents work via a variety of mechanisms, to affect cognition in a variety of different ways (Husain and Mehta, 2011).

Due to the high level of interest in providing novel nootropic compositions, there is an extensive patent literature on them. For example publication WO2014/037412 relates to compositions comprising at least two drugs selected form cinacalcet, baclofen, acamprosate, mexiletine, sulfisoxazole, and torasemide useful for enhancing memory and mental functions such as alertness, attention, reasoning, concentration, learning, or language processing in subjects.

Nevertheless it can be seen that characterising novel PCEs or nootropic substances to enhance cognition in health subjects would provide a contribution to the art.

### DISCLOSURE OF THE INVENTION

The present inventors have unexpectedly found that Leuco-methylthioninium acid salts (referred to herein as “LMTX” salts) can activate neuronal function at therapeutically relevant doses in normal (wild-type) animals. This is evidenced by an increase in basal acetylcholine (“ACh”) levels in hippocampus, and additionally by evidence of increased mean synaptophysin levels in various brain regions.

ACh is known to be important for cognitive function. Likewise, an increase in synaptophysin may enhance release of neurotransmitters which are needed to support cognitive and other mental functions.

The present findings imply new utilities for LMTX salts at therapeutically relevant doses for use as nootropics in healthy, non-impaired, subject-groups.

Bis(hydromethanesulfonate) (LMTM; USAN name hydromethylthionine mesylate) is being developed as a treatment targeting pathological aggregation of tau protein in AD (Wischik et al., 2018). The methylthioninium (MT) moiety can exist in oxidised (MT⁺) and reduced (LMT) forms. LMTM is a stabilised salt of LMT which has much better pharmaceutical properties than the oxidised MT⁺ form (Baddeley et al., 2015; Harrington et al., 2015). We have reported recently that LMT rather than MT⁺ is the active species blocking tau aggregation in vitro (Al-Hilaly et al., 2018). LMT blocks tau aggregation in vitro in cell-free and cell-based assays (Harrington et al., 2015; Al-Hilaly et al., 2018), and reduces tau aggregation pathology and associated behavioural deficits in tau transgenic mouse models in vivo at clinically relevant doses (Melis et al., 2015a). LMT also disaggregates the tau protein of the paired helical filaments (PHFs) isolated from AD brain tissues converting the tau into a form which becomes susceptible to proteases (Wischik et al., 1996; Harrington et al., 2015).

Although LMTM given orally produces brain levels sufficient for activity in vitro and in vivo (Baddeley et al., 2015), it had minimal apparent efficacy if taken as an add-on treatment in patients previously receiving symptomatic treatments in two large Phase 3 clinical trials (Gauthier et al., 2016; Wilcock et al., 2018). In subjects receiving LMTM as monotherapy, however, treatment produced marked slowing of cognitive and functional decline, reduction in rate of progression of brain atrophy measured by MRI and reduction in loss of glucose uptake measured by FDG-PET (Gauthier et al., 2016; Wilcock et al., 2018). When these outcomes were analysed in combination with population pharmacokinetic data available from subjects participating in the trials, LMTM was found to produce concentration-dependent effects whether taken alone or in combination with symptomatic treatments such as acetylcholinesterase inhibitors. However, the treatment effects in monotherapy subjects were substantially larger than in those taking LMTM after prior chronic treatment with symptomatic drugs approved for AD (acetylcholine esterase inhibitors and/or memantine).

LMTM and other Leuco-methylthioninium bis-protic acid salts have been suggested for the treatment of various diseases and pathologies in several publications e.g. WO2007/110627, WO2009/044127, WO2012/107706, WO2018019823 and WO2018041739.

However the findings of the present inventors have been made in wild-type animals showing no tau pathology, or other disease or impairment.

WO2008/155533 teaches the use of various diaminophenothiazines in the treatment of Mild Cognitive Impairment (MCI). MCI is discussed in the context of being a valid disease target by the FDA. It is defined by having a minor degree of cognitive impairment not yet meeting clinical criteria for a diagnosis of dementia. Hence the patient is neither normal nor demented. One patient group highlighted in WO2008/155533 is that having an Mini-Mental State Examination (MMSE) score of 24 to 29.

It is reported that the MT⁺ salt methylene blue (MB, Methyl Thioninium Chloride or MTC) undergoes redox cycling catalysed by complex I using NADH as co-factor whereby it accepts electrons which are subsequently transferred to complex IV. Thus it has been suggested to prevent or delay mitochondria-driven disorders (Atamna et al., 2012).

Several publications have suggested that MB may be used to enhance memory, in various contexts as described in those papers, which are typically impairment models. These include: Martinez et al (1978); Callaway et al. (2002); Gonzalez-Lima and Bruchey. (2004); Callaway et al. (2004); Riha et al. (2005); and Wrubel et al. (2007).

The implications that can be drawn from this art are discussed extensively in WO2008/155533.

However none of these publications teaches or suggest use of the compounds described herein in the claimed context.

A further more recent publication also suggests that MB may be used to enhance learning (Zoellner, et al., 2017).

In that study, during and shortly after treatment, there was apparently no MB benefit over placebo, although it was suggested that performance was improved a few months after treatment. Irrespective of this, the model used in the paper was one of posttraumatic stress disorder (PTSD).

Therefore this publication also does not teach or suggest use of the compounds described herein in the claimed context.

For a drug to act as a nootropic in healthy subjects, it must have a mechanism that permits its beneficial actions to occur in the absence of illness or the biochemical or physiological targets associated with that illness.

The present studies were undertaken with the aim of understanding the mechanisms responsible for the reduced efficacy of LMTM as an add-on to prior symptomatic treatments discussed above. In these studies a well-characterised tau transgenic mouse model (Line 1, “L1”; (Melis et al., 2015b)) was compared with wild-type mice.

One conclusion from the present studies is that homeostatic mechanisms downregulate multiple neuronal systems at different levels of brain function to compensate for the chronic pharmacological activation induced by prior symptomatic treatments. Compared with LMTM given alone, the effect of this downregulation is to reduce neurotransmitter release, levels of synaptic proteins, mitochondrial function and behavioural benefits if LMTM is given against a background of chronic prior exposure to acetylcholinesterase inhibitor. The behavioural benefits of LMTM are also reduced by prior chronic treatment with memantine.

Unexpectedly, however, the studies also revealed that LMTX salts can activate neuronal function even in non-impaired mice. As explained below these activating effects in relation to basal acetylcholine levels and synaptophysin release do not appear to be mediated by enhanced mitochondrial function or low-level acetyl cholinesterase inhibition.

The present findings imply new utilities for LMTX salts at therapeutically relevant doses for non-therapeutic use as nootropics in healthy, non-impaired, subject-groups.

Thus in one aspect there is provided non-therapeutic use of a methylthioninium (MT) containing compound to stimulate cognitive function in a healthy human subject,
wherein said use comprises orally administering between 2 and 100 mg of MT to the subject per day, optionally split into 2 or more doses,
wherein the MT compound is an LMTX compound of the following formula:

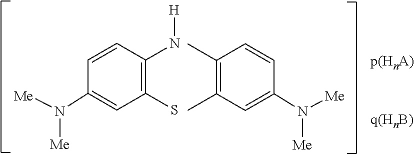

wherein each of HₙA and HₙB (where present) are protic acids which may be the same or different,
and wherein p=1 or 2; q=0 or 1; n=1 or 2; (p+q)×n=2.

In a further aspect there is provided non-therapeutic use of the LMTX compound to stimulate basal acetylcholine levels in a healthy subject, or to stimulate increased levels of the synaptic vesicular protein, synaptophysin. The latter indicates either more or larger vesicles required for release of a number of neurotransmitters (e.g. acetylcholine, noradrenaline, dopamine, glutamate, serotonin) in a healthy subject. This may be for the nootropic purposes described herein.

Another aspect of the present invention pertains to a non-therapeutic method of treating a healthy human subject to stimulate their cognitive function,
the treatment comprising administering to the subject a nootropically effective amount of methylthioninium (MT) containing LTMX compound as described herein, at a dosage as described herein.

Another aspect of the present invention pertains to a methylthioninium (MT) containing LTMX compound as described herein for use in a non-therapeutic method of treating a healthy human subject to stimulate their cognitive function, as described above.

Another aspect of the present invention pertains to use of a methylthioninium (MT) containing LTMX compound as described herein in the manufacture of a nootropic composition for stimulating cognitive function in a healthy human subject as described above.

The non-therapeutic stimulation of cognitive function may be for the purpose of stimulating (e.g., improve, enhance or increase) one or more memory and mental functions such as alertness, attention, reasoning, concentration, learning, or language processing in the healthy subject.

This in turn can be for more specific purposes e.g. to aid the ability to cope with a particular socio-professional burden in said subject.

The invention is suitable for non-therapeutic use in normal, non-demented (“healthy”) subjects, by which is meant those who have no known clinical signs of amnestic or cognitive impairment or disease. The subject may have other (physical or mental) impairments entirely unrelated to amnestic or cognitive impairment or disease.

The treatment is not for the relief or the amelioration of clinical amnestic symptoms or other cognitive impairment. Nor for the treatment of depression.

Subjects in relation to the present invention will be those who do not suffer from, and have not been diagnosed with e.g. vascular dementia, senile dementia, age-associated memory impairment, Alzheimer's disease, Lewy body dementia, Parkinson's disease or mild cognitive impairment). Such subjects may thus be diagnosed not to suffer from these diseases. Diagnosis in this context can be according to the generally recognized criteria of The Diagnostic and Statistical Manual of Mental Disorders, 5th edition (DSM-5, American Psychiatric Association, 2013).

Likewise such subjects will not suffer from PTSD or a defect in mitochondrial energy metabolism.

Such subjects may have an MMSE of 30.

The subjects may be those who are not receiving, and have not previously received, treatment with acetylcholinesterase inhibitors (AChEIs) or the N-methyl-D-aspartate receptor antagonist memantine. Examples of acetylcholinesterase inhibitors include Donepezil (Aricept™), Rivastigmine (Exelon™) or Galantamine (Reminyl™). An example of an NMDA receptor antagonist is Memantine (Ebixa™, Namenda™).

Such subjects may nevertheless have a desire for improved or stimulated cognitive capacities, either temporarily, or for longer periods of time.

For example the subject group may be entirely naïve to these other treatments, and have not historically received one or both of them.

However the subject group may have historically received one or both of these treatments, but ceased that medication at least 1, 2, 3, 4, 5, 6, 7 days, or 2, 3, 4, 5, 6, 7, 8, 12, or 16 weeks, or more preferably at least 1, 2, 3, 4, 5 or 6 months etc. prior to treatment with an MT compound according to the present invention.

Any aspect of the present invention may include the active step of selecting the subject group according to these criteria.

As explained in the Examples hereinafter, positive results were achieved in wild type NMRI mice at a dose of 5 mg/kg/day.

Based on the results herein, and prior and concurrent results using LMTM in the treatment of disease, it can be concluded that MT dosages in the range 2-80 or 100 mg/day could be beneficial for the nootropic effects described herein.

More specifically further analysis of the concentration-response for LMTM in relation to the treatment of disease supports the proposition that a preferred dose is at least 2 mg/day, and doses in the range 20-40 mg/day, or 20-60 mg/day would be expected to maximise the cognitive benefit while nevertheless maintaining a desirable profile in relation to being well tolerated with minimal side-effects. Since nootropics are indicated for healthy individuals, it is important that even rare adverse events or side-effects are minimised, and hence lower dosages may be preferred.

Thus in one embodiment, the total MT dose may be from around any of 2, 2.5, 3, 3.5, or 4 mg to around any of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 mg.

An example dosage is 2 to 60 mg e.g. 20, 30, 40, 50 or 60 mg.

An example dosage is 20 to 40 mg.

Further example dosages are 8 or 16 or 24 mg/day.

The subject of the present invention may be an adult human, and the dosages described herein are premised on that basis (typical weight 50 to 70 kg). If desired, corresponding dosages may be utilised for subjects outside of this range by using a subject weight factor whereby the subject weight is divided by 60 kg to provide the multiplicative factor for that individual subject.

As explained herein, in some embodiments the treatment will be a monotherapy, or at least will exclude prior administration of AChEIs or memantine.

Some of these aspects and embodiments will now be described in more detail:

### LMTX Compounds

Preferably the MT compound is an “LMTX” compound of the type described in WO2007/110627 or WO2012/107706.

Thus the compound may be selected from compounds of the following formula, or hydrates or solvates thereof:

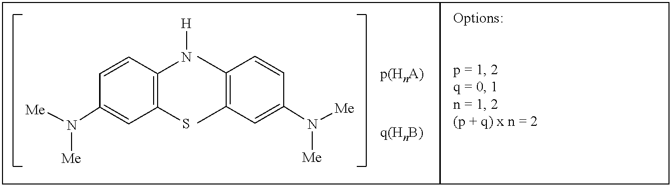

Each of HₙA and HₙB (where present) are protic acids which may be the same or different.

By “protic acid” is meant a proton (H+) donor in aqueous solution. Within the protic acid A⁻ or B⁻ is therefore a conjugate base. Protic acids therefore have a pH of less than 7 in water (that is the concentration of hydronium ions is greater than 10⁻⁷ moles per litre).

In one embodiment the salt is a mixed salt that has the following formula, where HA and HB are different mono-protic acids:

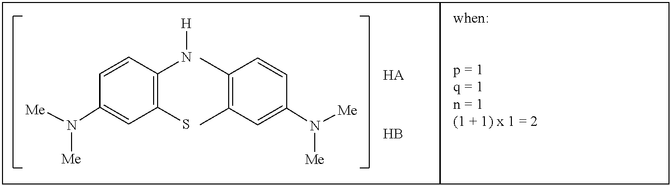

However preferably the salt is not a mixed salt, and has the following formula:

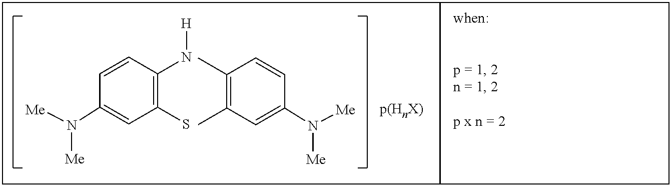

wherein each of HₙX is a protic acid, such as a di-protic acid or mono-protic acid.

In one embodiment the salt has the following formula, where H₂A is a di-protic acid:

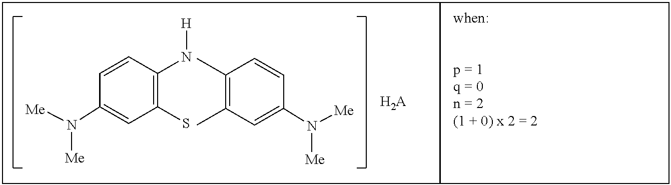

Preferably the salt has the following formula which is a bis monoprotic acid:

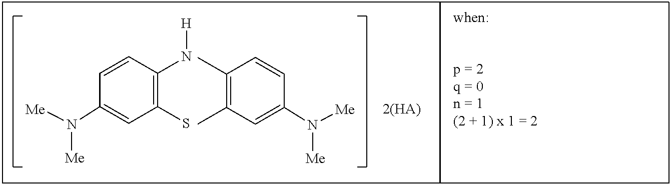

Examples of protic acids which may be present in the LMTX compounds used herein include:

Inorganic acids: hydrohalide acids (e.g., HCl, HBr), nitric acid (HNO₃), sulphuric acid (H₂SO₄)

Organic acids: carbonic acid (H₂CO₃), acetic acid (CH₃COOH), methanesulfonic acid, 1,2-ethanedisulfonic acid, ethanesulfonic acid, naphthalenedisulfonic acid, p-toluenesulfonic acid,

Preferred acids are monoprotic acid, and the salt is a bis(monoprotic acid) salt.

A preferred MT compound is LMTM:

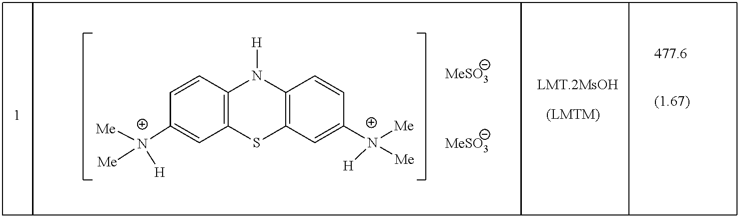

The anhydrous salt has a molecular weight of around 477.6. Based on a molecular weight of 285.1 for the LMT core, the weight factor for using this MT compound in the invention is 1.67. By “weight factor” is meant the relative weight of the pure MT containing compound vs. the weight of MT which it contains.

Other weight factors can be calculated for example MT compounds herein, and the corresponding dosage ranges can be calculated therefrom.

Therefore the invention embraces a total daily dose of around 0.8 to 33 mg/day of LMTM.

More preferably around 6 to 12 mg/day of LMTM total dose is utilised, which corresponds to about 3.5 to 7 mg MT.

Other example LMTX compounds are as follows. Their molecular weight (anhydrous) and weight factor is also shown:

| | | | |
|---|---|---|---|
| 2 | 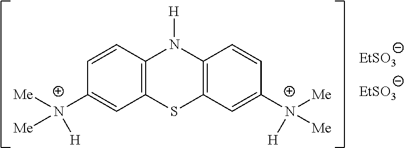 | LMT.2EsOH | 505.7 (1.77) |
| 3 | 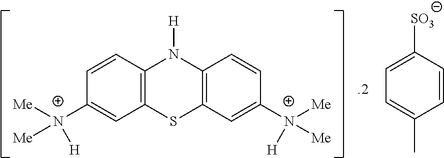 | LMT.2TsOH | 629.9 (2.20) |
| 4 | 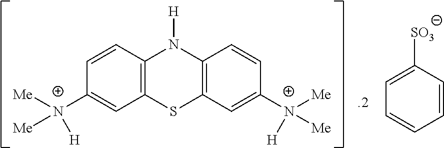 | LMT.2BSA | 601.8 (2.11) |
| 5 | 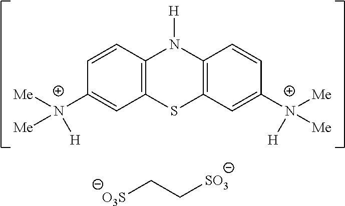 | LMT.EDSA | 475.6 (1.66) |
| 6 | 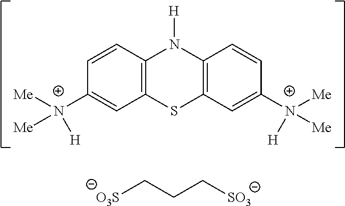 | LMT.PDSA | 489.6 (1.72) |
| 7 | 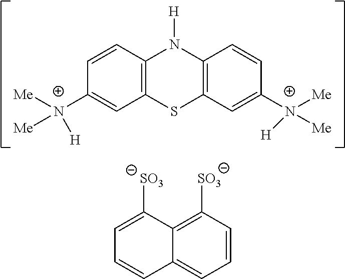 | LMT.NDSA | 573.7 (2.01) |
| 8 | 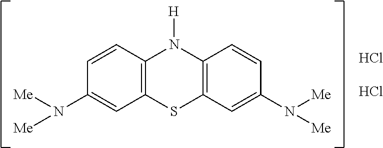 | LMT.2HCl | 358.33 (1.25) |

In the various aspects of the invention described herein (as they relate to an MT-containing compound) this may optionally be any of those compounds described above:

In one embodiment, it is compound 1.

In one embodiment, it is compound 2.

In one embodiment, it is compound 3.

In one embodiment, it is compound 4.

In one embodiment, it is compound 5.

In one embodiment, it is compound 6.

In one embodiment, it is compound 7.

In one embodiment, it is compound 8.

Or the compounds may be a hydrate, solvate, or mixed salt of any of these.

### Accumulation Factors

As will be appreciated by those skilled in the art, for a given daily dosage, more frequent dosing will lead to greater accumulation of a drug.

The present inventors have derived estimated accumulation factors for MT as follows:

| | Observed plasma | Relative |
|---|---|---|
| Dosing | accumulation for MT | accumulation |
| Once daily | 1.29^{extrapolated} | 1 |
| Twice daily | 1.47 | 1.13 |
| Three-times daily | 1.65 | 1.28 |

For example, considering a total daily dose of 3.5 to 7 mg MT:

When given as a single daily dose, this may equate to an accumulation of MT in plasma of 4.5 to 8

When split b.i.d., this may equate to an accumulation of MT in plasma of 5.1 to 10.3

When split t.i.d., this may equate to an accumulation of MT in plasma of 5.8 to 11.6

Therefore in certain embodiments of the invention, the total daily dosed amount of MT compound may be lower, when dosing more frequently (e.g. twice a day [b.i.d.] or three times a day [t.i.d.]).

In one embodiment, LMTM is administered around 9 mg/once per day; 4 mg b.i.d.; 2.3 mg t.i.d (based on weight of LMTM)

In one embodiment, LMTM is administered around 34 mg/once per day; 15 mg b.i.d.; 8.7 mg t.i.d (based on weight of LMTM)

### Combination Treatments

The term “treatment” includes “combination” non-therapeutic treatments, in which two or more treatments to stimulate cognitive function in a healthy subject (and/or to stimulate basal acetylcholine levels in a healthy subject and/or to increased levels of the synaptic vesicular protein synaptophysin indicating either more or larger vesicles required for release of a number of neurotransmitters in a healthy subject) are combined, for example, sequentially or simultaneously.

In combination treatments, the agents (i.e., an MT compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

An example of a combination treatment of the invention would be use of the MT compound with a nootropic previously known in the art.

Known nootropics belong to many different categories including traditional herbs, vitamins and supplements, recreational drugs, racetams, dopaminergics, serotonergics, anti-depressives, adaptogenic (antistress) and mood stabilization agents, vasodilators, antioxidants, neuroprotectant drugs, hormones, and other stimulants and concentration and memory enhancers.

The use of the MT compound in the methods or uses described herein in combination with any of these or other nootropics forms an aspect of the present invention.

In other embodiments the treatment is a “monotherapy”, which is to say that the MT-containing compound is not used in combination (within the meaning discussed above) with another active agent, whether a nootropic agent, or otherwise.

As noted above, it is specifically envisaged that administration of the MT-compound may be commenced in subjects who have not previously received (and are not currently receiving) with AChEIs or memantine.

However such AChEIs or memantine treatment may optionally be started or re-started after commencement of treatment with the MT compound, for example after at least or about 3 months of treatment with the MT compound.

### Oral Dosage Forms

The MT compound of the invention, or composition comprising it, is administered to a subject orally.

In some embodiments, the MT compound is administered as a nootropic composition comprising the LMTX compound as described herein, and a pharmaceutically acceptable carrier, diluent, or excipient.

The term “pharmaceutically acceptable,” as used herein, pertains to compounds, ingredients, materials, compositions, dosage forms, etc., which are suitable for use in contact with the tissues of the subject in question without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Compositions comprising LMTX salts are described in several publications e.g. WO2007/110627, WO2009/044127, WO2012/107706, WO2018019823 and WO2018041739.

In some embodiments, the composition is a nootropic composition comprising at least one LMTX compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In some embodiments, the composition further comprises other active nootropic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

In some embodiments, the composition is a dosage unit which is a tablet.

In some embodiments, the composition is a dosage unit which is a capsule.

In some embodiments, said capsules are gelatine capsules.

In some embodiments, said capsules are HPMC (hydroxypropylmethylcellulose) capsules.

In some embodiments, the amount of MT in the unit 2 to 60 mg.

In some embodiments, the amount of MT in the unit 10 to 40, or 10 to 60 mg.

In some embodiments, the amount of MT in the unit 20 to 40, or 20 to 60 mg.

An example dosage unit may contain 2 to 10 mg of MT.

A further example dosage unit may contain 2 to 9 mg of MT.

A further example dosage unit may contain 3 to 8 mg of MT.

A further preferred dosage unit may contain 3.5 to 7 mg of MT.

A further preferred dosage unit may contain 4 to 6 mg of MT.

In some embodiments, the amount is about 2, 2.5, 3, 3.5, 4, 5, 6, 7, 8, 9, 10, 11, 12, 13, 14, 15, 16, 17, 18, 19 or 20 mg of MT.

Using the weight factors described or explained herein, one skilled in the art can select appropriate amounts of an MT containing compound to use in oral formulations.

As explained above, the MT weight factor for LMTM is 1.67. Since it is convenient to use unitary or simple fractional amounts of active ingredients, non-limiting example LMTM dosage units may include about 3, 3.5, 4, 5, 6, 7, 8, 9, 10, 15, 16, 17, 34, 50, 63 mg etc.

The nootropic compositions described herein (e.g. a low dose MT containing compound plus optionally other ingredients) may be provided in a labelled packet along with instructions for their nootropic use.

In one embodiment, the pack is a bottle, such as are well known in the pharmaceutical art. A typical bottle may be made from pharmacopoeial grade HDPE (High-Density Polyethylene) with a childproof, HDPE push-lock closure and contain silica gel desiccant, which is present in sachets or canisters. The bottle itself may comprise a label, and be packaged in a cardboard container with instructions for us and optionally a further copy of the label.

In one embodiment, the pack or packet is a blister pack (preferably one having aluminium cavity and aluminium foil) which is thus substantially moisture-impervious. In this case the pack may be packaged in a cardboard container with instructions for us and label on the container.

Said label or instructions may provide information regarding the maximum permitted daily dosage of the compositions as described herein—for example based on once daily, b.i.d., or t.i.d.

Said label or instructions may provide information regarding the suggested duration of treatment.

### Salts and Solvates

Although the LMTX containing compounds described herein are themselves salts, they may also be provided in the form of a mixed salt (i.e., the compound of the invention in combination with another salt). Such mixed salts are intended to be encompassed by the term “and pharmaceutically acceptable salts thereof”. Unless otherwise specified, a reference to a particular compound also includes salts thereof.

The compounds of the invention may also be provided in the form of a solvate or hydrate. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, a penta-hydrate etc. Unless otherwise specified, any reference to a compound also includes solvate and any hydrate forms thereof.

Naturally, solvates or hydrates of salts of the compounds are also encompassed by the present invention.

As used herein the term “improvement” means an increment in memory, selective attention and/or performance in related mental functions when compared to a previous measure or reference data. Such performance in memory and/or memory related mental functions can be measured using several memory and cognition tests well known in the art.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting FIGURES and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1. Treatment effects of LMTM alone or following chronic pretreatment with rivastigmine in wild-type mice on hippocampal levels of acetylcholine (A) or synaptophysin levels measured immunohistochemically as the mean in hippocampus, visual cortex, diagonal band and septum (B). (**, p<0.01; ***, p<0.001).

### EXAMPLES

### Example 1—Provision of MT-Containing Compounds

Methods for the chemical synthesis of the MT-containing compounds described herein are known in the art. For example:

Synthesis of compounds 1 to 7 can be performed according to the methods described in WO2012/107706, or methods analogous to those.

Synthesis of compound 8 can be performed according to the methods described in WO2007/110627, or a method analogous to those.

### Example 2—Features of the Tau Transgenic Mouse Model Used for Interference Studies

In the L1 mouse model which was used in some of the present studies, there is over-expression of a three-repeat tau fragment encompassing residues 296-390 of the 2N4R tau isoform under the control of the Thy 1 promotor in an NMRI mouse strain (WO2002/059150). This fragment corresponds to the segment of tau first identified within the proteolytically stable core of the PHF (Wischik et al., 1988a; Wischik et al., 1988b) and recently confirmed by cryo-electronmicroscopy of PHFs in AD and tau filaments in Pick's disease (Fitzpatrick et al., 2017; Falcon et al., 2018).

Further features of the L1 mouse model include a prominent loss of neuronal immunoreactivity for choline acetyltransferase in the basal forebrain region, and a corresponding reduction in acetylcholinesterase in neocortex and hippocampus, indicative of reduction in acetylcholine. There is also an approximate 50% reduction in glutamate release for brain synaptosomal preparations from L1 mice compared with those from wild-type mice. In these respects, therefore, L1 mice also model the neurochemical impairments in cholinergic (Mesulam, 2013; Pepeu and Grazia Giovannini, 2017) and glutamatergic (Revett et al., 2013) function that are characteristic of AD.

Underlying these impairments in neurotransmitter function, the L1 mouse model shows a disturbance in integration of synaptic proteins. Quantitative immunohistochemistry for multiple synaptic proteins in the basal forebrain (vertical diagonal band) shows that there is normally a high degree of correlation in levels of proteins comprising the SNARE complex (e.g. SNAP-25, syntaxin, VAMP2; reviewed in Li and Kavalali, 2017), and the vesicular glycoprotein synaptophysin and α-synuclein in wild-type mice. These correlations are largely lost in L1 mice (Table 1). The only correlations that remain are between synaptophysin, syntaxin and VAMP2. Therefore, synaptic vesicular protein levels are no longer linked quantitatively to the proteins of the SNARE complex or α-synuclein. This suggests that the tau oligomer pathology of the L1 mice interferes with the functional integration between vesicular and membrane-docking proteins in the synapse.

### Example 3—Experimental Paradigms, Results and Discussion

### Experimental Paradigms

The treatment schedule used to study the negative interaction between symptomatic treatments and LMTM was designed to model the clinical situation in which subjects are first treated chronically with a cholinesterase inhibitor or memantine before receiving LMTM. In what follows, we summarise some of the key results obtained for the AChEI, rivastigmine. Wild-type and L1 mice (n=7-16 for each group) were pre-treated with rivastigmine (0.1 or 0.5 mg/kg/day) or memantine (2 or 20 mg/kg/day) or vehicle for 5 weeks by gavage. For the following 6 weeks, LMTM (5 and 15 mg/kg) or vehicle were added to this daily treatment regime, also by gavage. Animals were tested behaviourly during weeks 10 and 11 using a problem solving task in the open field water maze and then sacrificed for immunohistochemical and other tissue analyses.

Translating doses from mice to humans requires consideration of a number of factors. Although 5 mg/kg/day in mice corresponds approximately to 8 mg/day in humans in terms of Cₘₐₓ levels of parent MT in plasma, this dose is at the threshold for effects on pathology and behaviour. The higher dose of 15 mg/kg/day is generally required for LMTM to be fully effective in the L1 mouse model (Melis et al., 2015a). This may relate to the much shorter half-life of MT in mice (4 hours) compared to humans (37 hours in elderly humans). Tissue sectioned for immunohistochemistry was labelled with antibody and processed using Image J to determine protein expression densitometrically. Data are presented as Z-score transformations without units.

For measurement of acetylcholine (ACh) levels in hippocampus, animals (wild-type or L1) were treated with LMTM (5 mg/kg/day for 2 weeks) after prior treatment for 2 weeks with or without rivastigmine (0.5 mg/kg/day). Rivastigmine was administered subcutaneously with an Alzet minipump whereas LMTM was administered by oral gavage. Levels of ACh were measured in hippocampus using an implanted microdialysis probe and HPLC analysis of the extracellular fluid.

Data are presented as group averages and standard errors of mean and were analysed using parametric statistics, with alpha set to 0.05.

Experiments on animals were carried out in accordance with the European Communities Council Directive (63/2010/EU) with local ethical approval, a project license under the UK Scientific Procedures Act (1986), and in accordance with the German Law for Animal Protection (Tierschutzgesetz) and the Polish Law on the Protection of Animals.

### Results

Effects of Treatment with LMTM and Rivastigmine in Wild-Type Mice

The effects of treatment with LMTM alone or on a chronic rivastigmine background are summarised in Table 2.

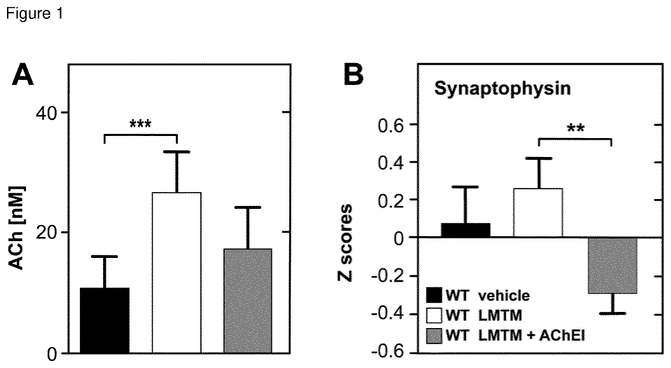

In wild-type mice, there was a significant, 2-fold increase in basal ACh levels in hippocampus following LMTM treatment, and a 30% reduction when mice received LMTM after prior treatment with rivastigmine (FIG. 1A).

There was also a 3-fold increase in mean synaptophysin levels measured in hippocampus, visual cortex, diagonal band and septum following LMTM treatment alone and a statistically significant reduction of the same magnitude when LMTM was given against a background of prior treatment with rivastigmine (FIG. 1B).

TABLE 2
Summary of treatment effects of LMTM given alone (5 or 15 mg/kg/day) or
following chronic pretreatment with rivastigmine (0.1 or 0.5 mg/kg/day) in
wild-type mice, given as approximate rounded percentages to
indicate scale and direction of change. Numbers in
black signify treatment effects which reached statistical significance,
those in grey were directional, ‘—’ indicates no effect.

| Effects in wild-type mice | LMTM alone | Rivastigmine + LMTM |
|---|---|---|
| ACh release | ↑ × 200% | ↓ × 30% |
| SNARE complex | — | — |
| Synaptophysin | ↑ × 300% | ↓ × 300% |
| α-Synuclein | — | — |
| Mitochondrial complex IV | — | — |
| Behaviour | — | — |

Effects of Treatment with LMTM and Rivastigmine in Tau Transgenic L1 Mice

The activating effects of LMTM alone and the inhibitory effects of the combination with rivastigmine are larger and more generalised in the tau transgenic L1 mice than in the wild-type mice (results not shown).

### Discussion of Example 3

The results presented here demonstrate that the reduction in efficacy of LMTM when given as an add-on to a symptomatic treatment in humans can be reproduced both in wild-type mice and in a tau transgenic mouse model.

The results we now report demonstrate that there are two classes of effect produced by LMTM treatment in wild-type and tau transgenic mice: those that are subject to dynamic modulation by prior exposure to cholinesterase inhibitor and those which are not. In tau transgenic mice, the treatment effects that can be modulated include increase in ACh release in the hippocampus, changes in synaptic proteins, increase in mitochondrial complex IV activity and reversal of behavioural impairment. The only treatment effects that are not subject to pharmacological modulation are the primary effect on tau aggregation pathology and its immediate effect on neuronal function, as measured for example by restoration of choline acetyltransferase expression in the basal forebrain.

Effects that are subject to pharmacological modulation are themselves of two types: those which are augmented by the effect on tau aggregation pathology and those which are also seen in wild-type mice. Of the outcomes we have measured, positive treatment effects of LMTM given alone in wild-type mice included an increase in ACh levels in hippocampus, and an increase in synaptophysin levels in multiple brain regions. Therefore, LMTM treatment is able to activate neuronal function at therapeutically relevant doses in wild-type mice lacking tau aggregation pathology.

In experimental models, cholinergic function is associated primarily with selective attention (Botly and De Rosa, 2007; 2008; Sarter et al., 2016), and the improvements in cognitive function resulting from cholinesterase inhibitors in AD are thought to be the result of elevated levels of acetylcholine in the synaptic cleft. However, these drugs are believed not to increase acetylcholine levels in wild-type mice because of efficient homeostatic adaptations which mitigate the inhibition of acetylcholinesterase inhibitors (e.g. by reducing levels of synaptic vesicles in the presynapse).

By contrast, LMTM does produce a significant increase in acetylcholine levels in the hippocampus, which is known to be important for cognitive function.

Likewise, an increase in synaptophysin signals an increase in number or size of the synaptic vesicles that are required for release of neurotransmitters from the presynapse following activation via an action potential. Therefore, an increase in synaptophysin levels appears to be associated with an increase in a number of neurotransmitters needed to support cognitive and other mental functions.

Although it has been reported that the MT moiety is a weak cholinesterase inhibitor (Pfaffendorf et al., 1997; Deiana et al., 2009), this is unlikely to be the mechanism responsible for the increase in ACh levels.

Specifically, further experiments using scopolamine to increase ACh levels (by blocking M2/M4 negative feedback receptors) showed that the increase produced by LMTM was less than that seen with rivastigmine alone, and that the combination was again inhibitory in wild type mice. Under the condition of cholinesterase inhibition used in these experiments (a very small amount of a cholinesterase inhibitor, 100 nanomolar rivastigmine, added to the perfusion fluid), ACh levels in the hippocampus rise, and when they rise strongly enough, they limit additional ACh release by activating pre-synaptic muscarinic receptors of the M2/M4 subtype (so-called negative feedback receptors).

In this situation, adding scopolamine (μM) to the perfusion fluid blocks these presynaptic receptors and, as a consequence, ACh levels rise by 3-5 fold. The fact that LMTM is not additive with rivastigmine in these experiments supports the conclusion that LMTM has a different mechanism of action from rivastigmine. In other words, although LMTM has been described as being a weak inhibitor of cholinesterases in high concentrations, the present effects seem to be unrelated to cholinesterase inhibition, because there is no additive effect with small quantities of rivastigmine.

The increase in ACh and synaptophysin levels might theoretically be explained by an increase in presynaptic mitochondrial activity, since the MT moiety is known to enhance mitochondrial complex IV activity (Atamna et al., 2012), and mitochondria have an important role in homeostatic regulation of presynaptic function (Devine and Kittler, 2018). In particular, The MT moiety is thought to enhance oxidative phosphorylation by acting as an electron shuttle between complex I and complex IV (Atamna et al., 2012). The MT moiety has a redox potential of approximately 0 mV, midway between the redox potential of complex I (−0.4 mV) and complex IV (+0.4 mV).

However, direct measurement of complex IV activity in wild type mice did not show any increase following LMTM treatment. The activating effects of LMTM were also not associated with improvement in spatial recognition memory in wild-type mice.

Chronic pretreatment with rivastagmine suppressed the cholinergic activation in the hippocampus and reduced synaptophysin levels more generally in the brains of wild-type mice. This effect is clearly not dependent on the effects of LMTM on tau aggregation pathology, since there is no pathology in wild-type mice. Rather, they point to a generalised homeostatic downregulation that counteracts the effect of combining two drugs which each have activating effects on neuronal function. Presumably, the primary mechanism that would normally protect against excessive levels of ACh in the synaptic cleft would be an increase in AChE activity. Since rivastigmine produces chronic impairment of this control system, pathways that would otherwise be activated by LMTM are suppressed in order to preserve homeostasis in cholinergic and other neuronal systems. Thus, LMTM-induced effects are subject to dynamic downregulation if the brain is already subject to chronic stimulation by a cholinesterase inhibitor.

A further consideration is whether the homeostatic downregulation that we have demonstrated would operate in the same way if LMTM treatment were primary and symptomatic treatment were added at a later date. The experiments we have conducted to date were originally designed to mimic the clinical situation in which LMTM is added in patients already receiving symptomatic treatments. If homeostatic downregulation is determined by the treatment that comes first, it is logical that the treatment effects of LMTM would dominate, albeit that the response to add-on symptomatic treatment could be reduced to some extent.

### REFERENCES

Al-Hilaly, Y. K., Pollack, S. J., Rickard, J. E., Simpson, M., Raulin, A.-C., Baddeley, T., et al. (2018). Cysteine-independent inhibition of Alzheimer's disease-like paired helical filament assembly by leuco-methylthioninium (LMT). J. Mol. Biol. 430, 4119-4131. doi: 10.1016/j.jmb.2018.08.010.
Atamna, H., Mackey, J., and Dhahbi, J. M. (2012). Mitochondrial pharmacology: electron transport chain bypass as strategies to treat mitochondrial dysfunction. Biofactors 38, 158-166. doi: 10.1002/biof.197.
Baddeley, T. C., Mccaffrey, J., Storey, J. M. D., Cheung, J. K. S., Melis, V., Horsley, D., et al. (2015). Complex disposition of methylthioninium redox forms determines efficacy in tau aggregation inhibitor therapy for Alzheimer's disease. J. Pharmacol. Exptl. Therapeutics 352, 110-118. doi: 10.1124/jpet.114.219352.
Botly, L. C., and De Rosa, E. (2007). Cholinergic influences on feature binding. Behav. Neurosci. 121, 264-276. doi: 10.1037/0735-7044.121.2.264.
Botly, L. C., and De Rosa, E. (2008). A cross-species investigation of acetylcholine, attention, and feature binding. Psychol. Sci. 19, 1185-1193. doi: 10.1111/j.1467-9280.2008.02221.x.
Callaway, N. L., Riha, P. D., Bruchey, A. K., Munshi, Z., and Gonzalez-Lima, F. (2004). Methylene blue improves brain oxidative metabolism and memory retention in rats. Pharmacol. Biochem. Behav. 77, 175-181.
Callaway, N. L., Riha, P. D., Wrubel, K. M., Mccollum, D., and Gonzalez-Lima, F. (2002). Methylene blue restores spatial memory retention impaired by an inhibitor of cytochrome oxidase in rats. Neurosci. Lett. 332, 83-86.
Deiana, S., Harrington, C. R., Wischik, C. M., and Riedel, G. (2009). Methylthioninium chloride reverses cognitive deficits induced by scopolamine: comparison with rivastigmine. Psychopharmacology 202, 53-65. doi: 10.1007/s00213-008-1394-2.
Devine, M. J., and Kittler, J. T. (2018). Mitochondria at the neuronal presynapse in health and disease. Nat. Rev. Neurosci. 19, 63-80. doi: 10.1038/nrn.2017.170.
Fitzpatrick, A. W. P., Falcon, B., He, S., Murzin, A. G., Murshudov, G., Garringer, H. J., et al. (2017). Cryo-EM structures of tau filaments from Alzheimer's disease. Nature 547, 185-190. doi: 10.1038/nature23002.
Gauthier, S., Feldman, H. H., Schneider, L. S., Wilcock, G. K., Frisoni, G. B., Hardlund, J. H., et al. (2016). Efficacy and safety of tau-aggregation inhibitor therapy in patients with mild or moderate Alzheimer's disease: a randomised, controlled, double-blind, parallel-arm, phase 3 trial. Lancet 388, 2873-2884. doi: 10.1016/S0140-6736(16)31275-2.
Gonzalez-Lima, F., and Bruchey, A. K. (2004). Extinction memory by the metabolic enhancer improvement methylene blue. Learning & Memory 11, 633-640.
Harrington, C. R., Storey, J. M. D., Clunas, S., Harrington, K. A., Horsley, D., Ishaq, A., et al. (2015). Cellular models of aggregation-dependent template-directed proteolysis to characterize tau aggregation inhibitors for treatment of Alzheimer's disease. J. Biol. Chem. 290, 10862-10875. doi: 10.1074/jbc.M114.616029.
Husain, M., and Mehta, M. A. (2011) Cognitive enhancement by drugs in health and disease. Trends Cognitive Sci. 15, 28-36.
Li, Y. and Kavalali, E. T. (2017). Synaptic vesicle-recycling machinery components as potential therapeutic targets. Pharmacol. Rev. 69, 141-160.
Maier, L. J., Ferris, J. A., and Winstock, A. R. (2018). Pharmacological cognitive enhancement among non-ADHD individuals—A cross-sectional study in 15 ountries.” Int. J. Drug Policy 58, 104-112.
Martinez, J. L., Jr., Jensen, R. A., Vasquez, B. J., Mcguiness, T., and Mcgaugh, J. L. (1978). Methylene blue alters retention of inhibitory avoidance responses. Physiological Psychology 6, 387-390.
Melis, V., Magbagbeolu, M., Rickard, J. E., Horsley, D., Davidson, K., Harrington, K. A., et al. (2015a). Effects of oxidized and reduced forms of methylthioninium in two transgenic mouse tauopathy models. Behav. Pharmacol. 26, 353-368. doi: 10.1097/fbp.0000000000000133.
Melis, V., Zabke, C., Stamer, K., Magbagbeolu, M., Schwab, K., Marschall, P., et al. (2015b). Different pathways of molecular pathophysiology underlie cognitive and motor tauopathy phenotypes in transgenic models for Alzheimer's disease and frontotemporal lobar degeneration. Cell. Mol. Life Sci. 72, 2199-2222. doi: 10.1007/s00018-014-1804-z.
Mesulam, M. M. (2013). Cholinergic circuitry of the human nucleus basalis and its fate in Alzheimer's disease. J. Comp. Neurol. 521, 4124-4144. doi: 10.1002/cne.23415.
Pepeu, G., and Grazia Giovannini, M. (2017). The fate of the brain cholinergic neurons in neurodegenerative diseases. Brain Res. 1670, 173-184. doi: 10.1016/j.brainres.2017.06.023.
Pfaffendorf, M., Bruning, T. A., Batink, H. D., and Van Zwieten, P. A. (1997). The interaction between methylene blue and the cholinergic system. Br. J. Pharmacol. 122, 95-98. doi: 10.1038/sj.bjp.0701355.
Revett, T. J., Baker, G. B., Jhamandas, J., and Kar, S. (2013). Glutamate system, amyloid beta peptides and tau protein: functional interrelationships and relevance to Alzheimer disease pathology. J. Psychiat. Neurosci. 38, 6-23. doi: 10.1503/jpn.110190.
Riha, P. D., Bruchey, A. K., Echevarria, D. J., and Gonzalez-Lima, F. (2005). Memory facilitation by methylene blue: Dose-dependent effect on behavior and brain oxygen consumption. Eur. J. Pharmacol. 511, 151-158.
Sarter, M., Lustig, C., Blakely, R. D., and Koshy Cherian, A. (2016). Cholinergic genetics of visual attention: Human and mouse choline transporter capacity variants influence distractibility. J. Physiol. (Paris) 110, 10-18. doi: 10.1016/j.jphysparis.2016.07.001.
Wilcock, G. K., Gauthier, S., Frisoni, G. B., Jia, J., Hardlund, J. H., Moebius, H. J., et al. (2018). Potential of low dose leuco-methylthioninium bis(hydromethanesulphonate) (LMTM) monotherapy for treatment of mild Alzheimer's disease: cohort analysis as modified primary outcome in a phase 3 clinical trial. J. Alzheimer's Dis. 61, 635-657. doi: 10.3233/JAD-170560.
Wischik, C. M., Edwards, P. C., Lai, R. Y. K., Roth, M., and Harrington, C. R. (1996). Selective inhibition of Alzheimer disease-like tau aggregation by phenothiazines. Proc. Natl. Acad. Sci. U.S.A. 93, 11213-11218. doi: 10.1073/pnas.93.20.11213.
Wischik, C. M., Novak, M., Edwards, P. C., Klug, A., Tichelaar, W., and Crowther, R. A. (1988a). Structural characterization of the core of the paired helical filament of Alzheimer disease. Proc. Natl. Acad. Sci. U.S.A. 85, 4884-4888. doi: 10.1073/pnas.85.13.4884.
Wischik, C. M., Novak, M., Thogersen, H. C., Edwards, P. C., Runswick, M. J., Jakes, R., et al. (1988b). Isolation of a fragment of tau derived from the core of the paired helical filament of Alzheimer disease. Proc. Natl. Acad. Sci. U.S.A. 85, 4506-4510. doi: 10.1073/pnas.85.12.4506.
Wischik, C. M., Schelter, B. O., Wischik, D. J., Storey, J. M. D., and Harrington, C. R. (2018). Modeling prion-like processing of tau protein in Alzheimer's disease for pharmaceutical development. J. Alzheimer's Dis. 62, 1287-1303. doi: 10.3233/JAD-170727.
Wrubel, K. M., Barrett, D., Shumake, J., Johnson, S. E., and Gonzalez-Lima, F. (2007). Methylene blue facilitates the extinction of fear in an animal model of susceptibility to learned helplessness. Neurobiol. Learning Memory 87, 209-217.
Zoellner, L. A., Telch, M., Foa, E. B., Farach, F. J., Mclean, C. P., Gallop, R., et al. (2017). Enhancing extinction learning in posttraumatic stress disorder wWith brief daily imaginal exposure and methylene blue: a randomized controlled trial. J. Clin. Psychiat. 78, e782-e789. doi: 10.4088/JCP.16m10936.

1. Non-therapeutic use of a methylthioninium (MT) containing compound to stimulate cognitive function in a healthy subject,
wherein said use comprises administering between 2 and 100 mg of MT to the subject per day, optionally split into 2 or more doses,
wherein the MT compound is an LMTX compound of the following formula: 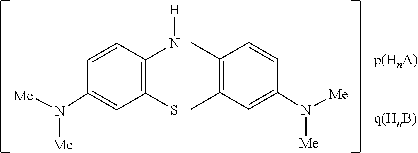
wherein each of HₙA and HₙB (where present) are protic acids which may be the same or different,
and wherein p=1 or 2; q=0 or 1; n=1 or 2; (p+q)×n=2
or a hydrate or solvate thereof.

2. The non-therapeutic use of claim 1 wherein the LMTX compound stimulate basal choline levels and synaptophysin in the subject.

3. The non-therapeutic use as claimed in claim 1 or claim 2 wherein the stimulation of cognitive function is for the purpose of stimulating alertness, attention, reasoning, concentration, learning, or language processing in the subject.

4. The non-therapeutic use as claimed in any one of claims 1 to 3 wherein the subject has an MMSE of 30.

5. The non-therapeutic use as claimed in any one of claims 1 to 4 wherein the subject is has not previously received, treatment with an acetylcholinesterase inhibitor or an N-methyl-D-aspartate receptor antagonist, or has discontinued such treatment prior to administration of the LMTX compound.

6. The non-therapeutic use as claimed in any one of claims 1 to 5 wherein the total daily dose of MT is from 10-60 mg.

7. The non-therapeutic use as claimed in any one of claims 1 to 6 wherein the total daily dose of MT is from 20-60 mg.

8. The non-therapeutic use as claimed in any one of claims 1 to 6 wherein the total daily dose of MT is from 10-40 mg.

9. The non-therapeutic use as claimed in any one of claims 1 to 8 wherein the total daily dose of MT is from 20-40 mg.

10. The non-therapeutic use as claimed in any one of claims 1 to 5 wherein the total daily dose of MT is from around any of 2, 2.5, 3, 3.5, or 4 mg to around any of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50 or 60 mg.

11. The non-therapeutic use as claimed in any one of claims 1 to 10 wherein the total daily dose of the compound is administered as a split dose twice a day or three times a day.

12. The non-therapeutic use as claimed in any one of claims 1 to 11 wherein the compound has the following formula, where HA and HB are different mono-protic acids: 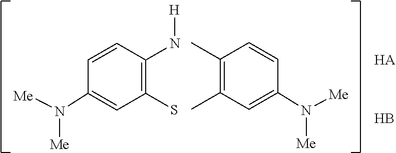
wherein HA and HB are different mono-protic acids:

13. The non-therapeutic use as claimed in any one of claims 1 to 11 wherein the compound has the following formula: 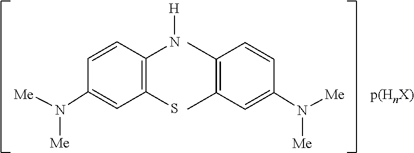
wherein each of HₙX is a protic acid.

14. The non-therapeutic use as claimed in any one of claims 1 to 11 wherein the compound has the following formula and H₂A is a di-protic acid:

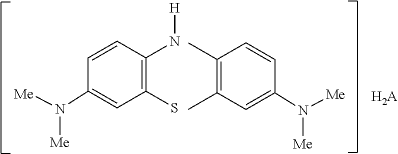

15. The non-therapeutic use as claimed in claim 13 wherein the compound has the following formula and is a bis-monoprotic acid:

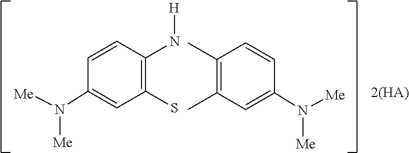

16. The non-therapeutic use as claimed in any one of claims 1 to 15 wherein the or each protic acid is an inorganic acid.

17. The non-therapeutic use as claimed in claim 16 wherein each protic acid is a hydrohalide acid.

18. The non-therapeutic use as claimed in claim 16 wherein the or each protic acid is selected from HCl; HBr; HNO₃; H₂SO₄.

19. The non-therapeutic use as claimed in any one of claims 1 to 15 wherein the or each protic acid is an organic acid.

20. The non-therapeutic use as claimed in claim 19 wherein the or each protic acid is selected from H₂CO₃; CH₃COOH; methanesulfonic acid, 1,2-ethanedisulfonic acid, ethanesulfonic acid, naphthalenedisulfonic acid, p-toluenesulfonic acid.

21. The non-therapeutic use as claimed in claim 20 wherein the compound is LMTM:

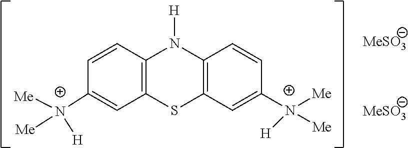

22. The non-therapeutic use as claimed in claim 21 wherein the total daily dose of LMTM is around 3.4 to 100 mg/day, more preferably 34 to 100 mg/day of LMTM total.

23. The non-therapeutic use as claimed in claim 22 wherein the dose of LMTM is around 34 mg/once per day; 15 mg b.i.d.; 8.7 mg t.i.d.

24. The non-therapeutic use as claimed in claim 20 wherein the compound is selected from the list consisting of:

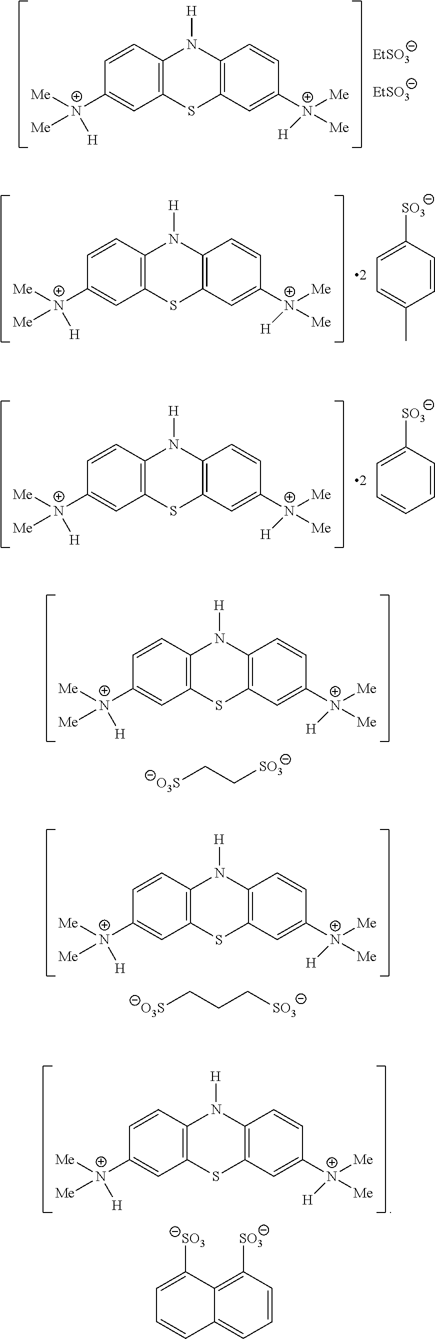

25. The non-therapeutic use as claimed in any one of claims 1 to 24 wherein the MT compound is provided as a nootropic composition comprising the MT compound and a pharmaceutically acceptable carrier or diluent, optionally in the form of a dosage unit.

26. The non-therapeutic use as claimed in claim 25 wherein the amount of MT in the unit is about 4, 5, 6, 7, 8, 9, 10, 20, or 30 to about 40, 50 or 60 mg.

27. The non-therapeutic use as claimed in any one of claims 1 to 26 wherein the MT compound is provided as a nootropic composition comprising the MT compound and a further nootropic agent, optionally in the form of a dosage unit.

28. The non-therapeutic use as claimed in claim 25 or claim 26 which is a tablet or capsule.

29. A container comprising:
(i) a plurality of dosage units as defined in any of claims 25 to 28;
(ii) a label and\or instructions for their non-therapeutic use as defined in any one of claims 1 to 24.

30. A container as claimed in claim 29, wherein the dosage units are present in a blister pack which is substantially moisture-impervious.

31. A container as claimed in claim 29 or claim 30 wherein the label or instructions provide information regarding the stimulation of cognitive function for which the composition is intended.

32. A container as claimed in any one of claims 29 to 31 wherein the label or instructions provide information regarding the maximum permitted daily dosage of the dosage units.

33. A non-therapeutic method of treating a healthy human subject to stimulate their cognitive function,
the treatment comprising orally administering to the subject a nootropically effective amount of methylthioninium (MT) containing LTMX compound according to the use defined in any one of claims 1 to 28.

34. A methylthioninium (MT) containing LTMX compound as defined in any one of claims 1 to 28 for use in a non-therapeutic method of treating a healthy human subject to stimulate their cognitive function according to the use defined in any one of claims 1 to 28.

35. Use of a methylthioninium (MT) containing LTMX compound as defined in any one of claims 1 to 28 in the manufacture of a nootropic composition for stimulating cognitive function in a healthy human subject according to the use defined in any one of claims 1 to 28.
